(19) United States
(12) Patent Application Publication
(10) Pub. No.: US 2025/0143600 A1
(43) Pub. Date: May 8, 2025

(54) SYSTEM AND METHOD FOR GENERATING ALERT BASED ON MONITORING OF NECK ALIGNMENT OF USER

(71) Applicant: Samsung Electronics Co., Ltd., Suwon-si (KR)

(72) Inventors: Jawwad AKHTER, Noida (IN); Ayan TALUKDAR, Noida (IN)

(21) Appl. No.: 18/804,703

(22) Filed: Aug. 14, 2024

Related U.S. Application Data
(63) Continuation of application No. PCT/KR2024/007463, filed on May 31, 2024.

(30) Foreign Application Priority Data
Nov. 8, 2023 (IN) 202311076456

(51) Int. Cl. A61B 5/11; A61B 5/00

(52) U.S. Cl. CPC A61B 5/1116; A61B 5/1114; A61B 5/1121; A61B 5/486; A61B 5/6803; A61B 5/7282; A61B 5/746

(57) ABSTRACT

A method for generating an alert based on monitoring of neck alignment of a user is provided. The method includes determining via one or more sensors of a wearable device worn by the user, an orientation of a head of the user based on an inclination of at least one sensor of the one or more sensors, evaluating a tilt angle associated with the head based on the determined orientation, determining a degree of misalignment of a neck of the user based on the tilt angle, determining a score associated with a posture of the neck based on a body mass index of the user, the tilt angle, a force on the neck, and the degree of misalignment of the neck, determining, based on the score, whether the posture corresponds to a specific type, and generating the alert, in response to determining that the posture corresponds to the specific type.

### CROSS-REFERENCE TO RELATED APPLICATION(S)

This application is a continuation application, claiming priority under § 365 (c), of an International application No. PCT/KR2024/007463, filed on May 31, 2024, which is based on and claims the benefit of an Indian Patent Application number 202311076456, filed on Nov. 8, 2023, in the Indian Intellectual Property Office, the disclosure of which is incorporated by reference herein in its entirety.

### TECHNICAL FIELD

The disclosure relates to generating an alert based on monitoring. More particularly, the disclosure relates to a method and a system for generating an alert based on monitoring of neck alignment of a user using a wearable device.

### BACKGROUND

Neck pain is one of the most common musculoskeletal disorders (MSDs) experienced by a lot of people in their day-to-day lives. Poor neck alignment may cause a range of problems, including neck pain, headaches, and even chronic issues like arthritis, thus adversely affecting both physical and mental well-being. Besides, continuously maintaining poor neck alignment may lead to gradual deterioration of neck posture and may have major effects on the cervical vertebrae.

Further, abnormal or flexed head posture has a significant effect on the entire body especially on different vertebrae of the cervical spine, e.g., the neck area of a spine. Bad sitting postures and angle of head bending exert pressure on the cervical spine causing back and neck pain. Unawareness and ignorance of poor neck alignments may lead to fatal consequences such as permanent damage to the neck alignment and, cervical instability connection on the neck. Over a prolonged period of time, poor neck alignments may lead to problems like spondylosis.

In addition to chronic neck pain caused by poor neck alignment, involuntary accidents in the cervical spine, such as choking or neck spasms, may also occur. Such accidents are often a result of the strain and vulnerability created by bad neck alignment. Thus, maintaining a good neck alignment is important for maintaining a healthy spine, and for the overall health and well-being of an individual.

Related arts for maintaining a good neck alignment majorly rely on hardware components for implementation and maintenance. The hardware components include specific devices and equipment such as an array of sensors attached to the head and neck, which are utilized to compute the movement of the head and the neck, one or more camera systems equipped with multiple force sensors, multiple inertial measuring devices, and optical tracking camera system. Such specific devices and equipment limit accessibility and compatibility across different platforms.

Therefore, there lies a need for an improved method and system that can overcome the above-described limitations and problems associated with poor neck alignment.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a method, a system, and a computer-readable storage medium (e.g., non-transitory computer-readable storage medium) for generating an alert based on monitoring of neck alignment of a user using a wearable device.

Additional aspects will be set forth in part in the description which follows and, in part, will be apparent from the description, or may be learned by practice of an embodiment of the disclosure.

In accordance with an aspect of the disclosure, a method performed by a wearable device for generating an alert based on monitoring of neck alignment of a user is provided. The method may include determining, by the wearable device, via one or more sensors of the wearable device worn by the user, an orientation of a head of the user based on an inclination of at least one sensor of the one or more sensors. The method may include evaluating, by the wearable device, a tilt angle associated with the head based on the determined orientation. The method may include determining, by the wearable device, a degree of misalignment of a neck of the user based on the tilt angle. The method may include determining, by the wearable device, a score associated with a posture of the neck based on a body mass index (BMI) of the user, the tilt angle, a force on the neck, and the degree of misalignment of the neck. The method may include determining, by the wearable device, based on the score, whether the posture corresponds to a specific type. The method may include generating, by the wearable device, the alert, in response to determining that the posture corresponds to the specific type.

In accordance with an aspect of the disclosure, a wearable device for generating an alert based on monitoring of neck alignment of a user is provided. The wearable device may include one or more sensors. The wearable device may include memory storing one or more computer programs. The wearable device may include one or more processors communicatively coupled to the one or more sensors and the memory, wherein the one or more computer programs include computer-executable instructions that, when executed by the one or more processors, cause the wearable device to determine, via the one or more sensors of the wearable device worn by the user, an orientation of a head of the user based on an inclination of at least one sensor of the one or more sensors. The one or more processors may cause the wearable device to evaluate a tilt angle associated with the head based on the determined orientation. The one or more processors may cause the wearable device to determine a degree of misalignment of the neck based on the tilt angle. The one or more processors may cause the wearable device to determine a score associated with a posture of the neck based on a body mass index (BMI) of the user, the tilt angle, a force on the neck, and the degree of misalignment of the neck. The one or more processors may cause the wearable device to determine, based on the score, whether the posture corresponds to a specific type. The one or more processors may cause the wearable device to generate the alert, in response to determining that the posture corresponds to the specific type.

In accordance with an aspect of the disclosure, one or more computer-readable storage media storing one or more computer programs including computer-executable instructions that, when executed by one or more processors of a wearable device, cause the wearable device to perform operations are provided. The operations may include determining, by the wearable device, via one or more sensors of the wearable device worn by a user, an orientation of a head of the user based on an inclination of at least one sensor of the one or more sensors. The operations may include evaluating, by the wearable device, a tilt angle associated with the head based on the determined orientation. The operations may include determining, by the wearable device, a degree of misalignment of a neck of the user based on the tilt angle. The operations may include determining, by the wearable device, a score associated with a posture of the neck based on a body mass index (BMI) of the user, the tilt angle, a force on the neck, and the degree of misalignment of the neck. The operations may include determining, by the wearable device, based on the score, whether the posture corresponds to a specific type. The operations may include generating, by the wearable device, an alert, in response to determining that the posture corresponds to the specific type.

In accordance with an aspect of the disclosure, a computer-readable storage medium storing instructions is provided. The instructions, when executed by at least one processor, may cause the at least one processor to perform the method corresponding.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of an embodiment of the disclosure will be more apparent from the following description taken in conjunction with the accompanying drawings, in which:

FIG. 1 illustrates a pictorial diagram depicting an environment for implementing a system for generating the alert based on monitoring of neck alignment of a user, according to an embodiment of the disclosure;

FIG. 2 illustrates a block diagram depicting the system for generating the alert based on monitoring of the neck alignment of the user 101, according to an embodiment of the disclosure;

FIG. 3 illustrates a block diagram depicting the modules, according to an embodiment of the disclosure;

FIG. 4 illustrates a block diagram depicting an operational flow of the modules, according to an embodiment of the disclosure;

FIG. 5 illustrates a schematic diagram depicting an orientation of a user's neck and head, according to an embodiment of the disclosure;

FIGS. 6A and 6B illustrate graphical diagrams depicting the relationship among head tilt angle, the gravitational force on the neck, and the neck tilt angle, according to an embodiment of the disclosure;

FIG. 7 illustrates a pictorial diagram depicting an impact of the score associated with the posture of the neck on the sections of the set of cervical vertebrae, according to an embodiment of the disclosure; and

FIG. 8 illustrates a flow diagram depicting the method for generating the alert based on monitoring of neck alignment of the user, according to an embodiment of the disclosure.

The same reference numerals are used to represent the same elements throughout the drawings.

### DETAILED DESCRIPTION

The following description with reference to the accompanying drawings is provided to assist in a comprehensive understanding of an embodiment of the disclosure as defined by the claims and their equivalents. It includes various specific details to assist in that understanding but these are to be regarded as merely exemplary. Accordingly, those of ordinary skill in the art will recognize that various changes and modifications of an embodiment described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of an embodiment of the disclosure is provided for illustration purpose only and not for the purpose of limiting the disclosure as defined by the appended claims and their equivalents.

It is to be understood that the singular forms “a”, “an”, and “the” include plural referents unless the context clearly dictates otherwise. Thus, for example, reference to “a component surface” includes reference to one or more of such surfaces.

As used here, terms and phrases such as “have”, “may have”, “include”, or “may include” a feature (like a number, function, operation, or component such as a part) indicate the existence of the feature and do not exclude the existence of other features. Also, as used here, the phrases “A or B”, “at least one of A and/or B”, or “one or more of A and/or B” may include all possible combinations of A and B. For example, “A or B”, “at least one of A and B”, and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of the disclosure.

It will be understood by those skilled in the art that the foregoing general description and the following detailed description are explanatory of the disclosure and are not intended to be restrictive thereof.

Reference throughout this specification to “an aspect”, “another aspect” or similar language means that a particular feature, structure, or characteristic described in connection with the embodiment is included in at least one embodiment of the disclosure. Thus, appearances of the phrase “in an embodiment”, “in another embodiment” and similar language throughout this specification may, but do not necessarily, all refer to the same embodiment.

It is to be understood that as used herein, terms such as, “includes”, “comprises”, “has”, etc. are intended to mean that the one or more features or elements listed are within the element being defined, but the element is not necessarily limited to the listed features and elements, and that additional features and elements may be within the meaning of the element being defined. In contrast, terms such as, “consisting of” are intended to exclude features and elements that have not been listed.

The embodiments herein and the various features and advantageous details thereof are explained more fully with reference to the non-limiting embodiments that are illustrated in the accompanying drawings and detailed in the following description. Descriptions of well-known components and processing techniques are omitted to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

As is traditional in the field, embodiments may be described and illustrated in terms of blocks that carry out a described function or functions. These blocks, which may be referred to herein as units or modules or the like, are physically implemented by analog or digital circuits such as logic gates, integrated circuits, microprocessors, microcontrollers, memory circuits, passive electronic components, active electronic components, optical components, hardwired circuits, or the like, and may optionally be driven by firmware and software. The circuits may, for example, be embodied in one or more semiconductor chips, or on substrate supports such as printed circuit boards and the like. The circuits constituting a block may be implemented by dedicated hardware, by a processor (e.g., one or more programmed microprocessors and associated circuitry), or by a combination of dedicated hardware to perform some functions of the block and a processor to perform other functions of the block. Each block of the embodiments may be physically separated into two or more interacting and discrete blocks without departing from the scope of the disclosure. Likewise, the blocks of the embodiments may be physically combined into more complex blocks without departing from the scope of the disclosure.

The accompanying drawings are used to help easily understand various technical features and it should be understood that the embodiments ed herein are not limited by the accompanying drawings. As such, the disclosure should be construed to extend to any alterations, equivalents, and substitutes in addition to those which are particularly set out in the accompanying drawings. Although the terms first, second, etc. may be used herein to describe various elements, these elements should not be limited by these terms. These terms are generally only used to distinguish one element from another.

An object of the disclosure is to provide an improved technique to overcome the above-described limitations associated with poor neck alignment. The object of the disclosure is to provide a technique for maintaining a good neck alignment that, unlike existing solutions, does not rely heavily on various hardware components.

The disclosure achieves the above-described objectives by providing a technique to monitor the neck alignment of the user without having to rely on various hardware components. The disclosed technique provides neck alignment correction and recommendations based on the movement angle of the user's neck.

The disclosed techniques relate to a system and method for generating the alert based on monitoring of neck alignment of the user using a wearable device, as described below in the forthcoming paragraphs.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

The processor may include various processing circuitry and/or multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

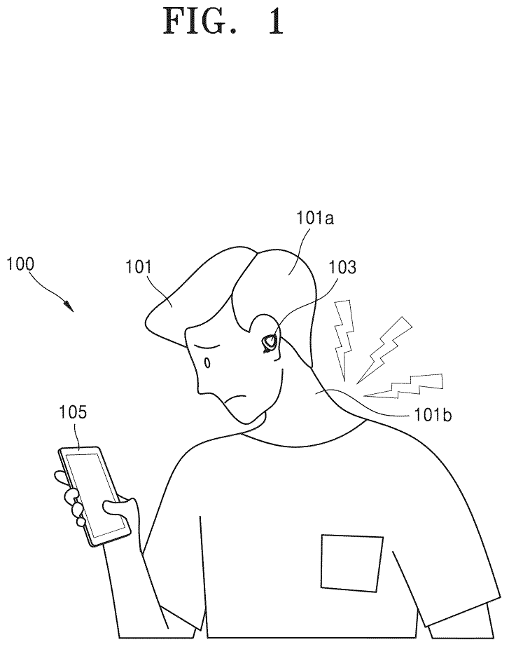

FIG. 1 illustrates a pictorial diagram depicting an environment 100 for implementing a system for generating the alert based on monitoring of neck alignment of a user, according to an embodiment of the disclosure.

Referring to FIG. 1, the environment 100 may include a user 101 using a wearable device 103. The wearable device 103 may include at least one of smart earphones, smart headphones, smart glasses, or any other smart wearable device that can be worn around the head 101a of the user 101.

The wearable device 103 may be communicatively coupled with a user device 105. The user device 105 may include a smartphone, a tablet, a laptop, a smart television, a smartwatch, or any other device that can be communicatively coupled with the wearable device 103.

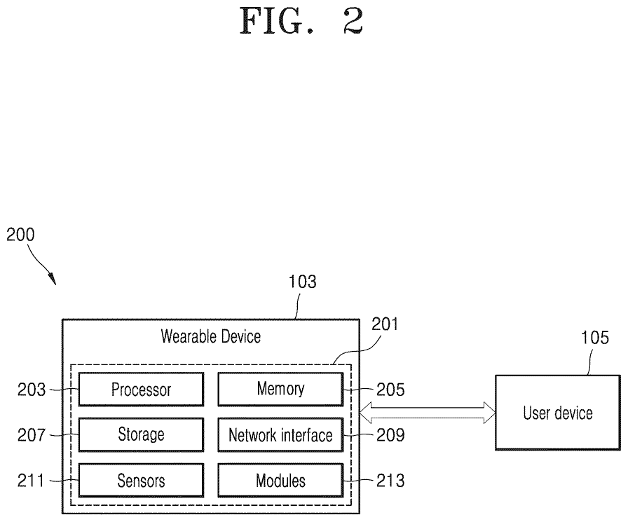

In an embodiment, a system 201 (as shown in FIG. 2) for generating the alert based on monitoring of neck alignment of the user 101 may be employed within the wearable device 103. The neck 101b, scientifically known as the cervical spine, is responsible for supporting the weight of the head 101a, facilitating movement, and protecting the delicate spinal cord that runs through the cervical spine. The neck or the cervical spine consists of seven vertebrae C1 through C7, where each vertebra corresponds to an individual bone that makes up a vertebral column of the cervical spine.

In an embodiment of the disclosure, an orientation of the head 101a of the user 101 is determined based on an inclination of at least one sensor of one or more sensors of the wearable device 103 worn by the user 101. Based on the determined orientation, a tilt angle associated with the head 101a is evaluated. A degree of misalignment of the neck 101b of the user is determined based on the tilt angle. A score associated with a posture of the neck 101b is determined. Based on the score, a poor or average posture of the neck 101b is determined. An alert is generated. In response to generating the alert, one or more corrective actions are recommended to the user 101. The system 201 is now described below in greater detail in conjunction with FIG. 2.

FIG. 2 illustrates a block diagram depicting a system for generating an alert based on monitoring of a neck alignment of a user, according to an embodiment of the disclosure.

Referring to FIG. 2, in a block diagram 200, a system 201 may include at least one processor 203, memory 205, a storage 207, a network interface 209, one or more sensors 211, and module(s) 213 coupled with each other.

In an example, the at least one processor 203 is a single processing unit or a number of units, all of which could include multiple computing units. The at least one processor 203 may be implemented as one or more microprocessors, microcomputers, microcontrollers, digital signal processors, central processing units, logical processors, virtual processors, state machines, logic circuitries, and/or any devices that manipulate signals based on operational instructions. Among other capabilities, the at least one processor 203 is configured to fetch and execute computer-readable instructions and data stored in the memory 205.

The memory 205 may include any computer-readable medium known in the art including, for example, volatile memory, such as static random access memory (SRAM) and dynamic random access memory (DRAM), and/or non-volatile memory, such as read-only memory (ROM), erasable programmable ROM, flash memories, hard disks, optical disks, and magnetic tapes.

As an example, the storage 207 may be implemented with integrated hardware and software. The hardware may include a hardware disk controller with programmable search capabilities or a software system running on general-purpose hardware. The examples of the storage 207 include, but are not limited to, in-memory databases, cloud databases, distributed databases, embedded databases, and the like. The storage 207, amongst other things, serves as a repository for storing data processed, received, and generated by one or more of the processors, and the modules/engines/units.

The network interface 209 may be configured to provides network connectivity and enables communication with paired devices such as the user device 105. The network connectivity may be provided via a wireless connection or a wired connection. For example, the network connectivity is provided via cellular technology, such as third generation (3G), fourth generation (4G), fifth generation (5G), pre-5G, sixth generation (6G), or any other wireless communication technology such as Bluetooth.

The one or more sensors 211 may include, but are not limited to, an inertial measurement unit (IMU). The IMU may refer to a combination of accelerometers and gyroscopes to measure the device's motion and orientation in three-dimensional space. According to an embodiment of the disclosure, data obtained from the one or more sensors 211 may be utilized to determine an orientation of the head 101a of the user 101 based on an inclination of at least one sensor of the one or more sensors 211.

As an example, the module(s) 213 may include a program, a subroutine, a portion of a program, a software component, or a hardware component capable of performing a stated task or function. As used herein, the module(s) 213 may be implemented on a hardware component such as a server independently of other modules, or a module can exist with other modules on the same server, or within the same program. The module(s) 213 may be implemented on a hardware component, such as processor one or more microprocessors, microcomputers, microcontrollers, digital signal processors, central processing units, state machines, logic circuitries, and/or any devices that manipulate signals based on operational instructions. The module(s) 213 when executed by the processor(s) 203 may be configured to perform any of the functionalities discussed herein.

In an embodiment, the module(s) 213 may be implemented using one or more artificial intelligence (AI) modules that may include a plurality of neural network layers. Examples of neural networks include but are not limited to, Convolutional Neural Network (CNN), Deep Neural Network (DNN), Recurrent Neural Network (RNN), and Restricted Boltzmann Machine (RBM). Further, ‘learning’ may be referred to in the disclosure as a method for training a predetermined target device (e.g., a robot) using a plurality of learning data to cause, allow, or control the target device to make a determination or prediction. Examples of learning techniques include, but are not limited to supervised learning, unsupervised learning, semi-supervised learning, or reinforcement learning. At least one of a plurality of CNN, DNN, RNN, RMB models and the like may be implemented to thereby achieve execution of the subject matter's mechanism through an AI model. A function associated with an AI module may be performed through the non-volatile memory, the volatile memory, and the processor. The processor may include one or a plurality of processors. At this time, one or a plurality of processors may be a general-purpose processor, such as a central processing unit (CPU), an application processor (AP), or the like, a graphics-only processing unit such as a graphics processing unit (GPU), a visual processing unit (VPU), and/or an AI-dedicated processor, such as a neural processing unit (NPU). One or a plurality of processors control the processing of the input data in accordance with a predefined operating rule or artificial intelligence (AI) model stored in the non-volatile memory and the volatile memory. The predefined operating rule or artificial intelligence model is provided through training or learning

In an embodiment, the system 201 may be implemented completely in the wearable device 103. In an embodiment, the system may be implemented in a distributed manner such that one or more components of the system 201 are implemented on the wearable device 103 and one or more components are implemented on the user device 105.

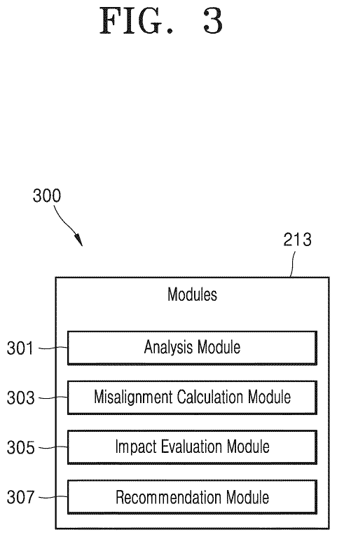

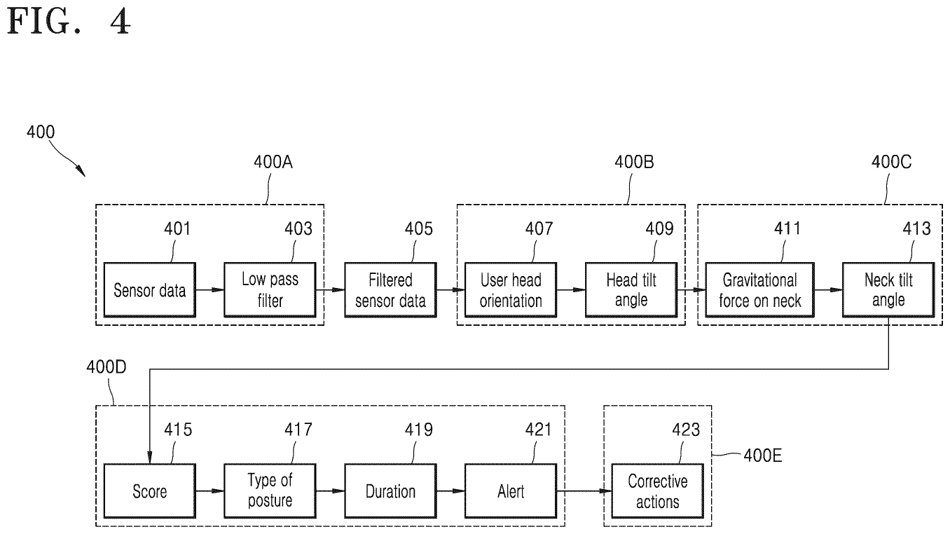

The modules 213 may include a set of instructions that may be executed to cause the wearable device 103 to monitor the neck alignment of the user 101. The modules 213 and operational flow of the modules are described below in detail in conjunction with FIGS. 3 and 4.

FIG. 3 illustrates a block diagram depicting modules, according to an embodiment of the disclosure. FIG. 4 illustrates a block diagram depicting an operational flow of modules 213 and hence will be explained with FIG. 3 for the sake of brevity and ease of reference. Further, the reference numerals were kept the same for the similar components throughout the disclosure for ease of explanation and understanding.

Referring to FIG. 3, in a block diagram 300, modules 213 may include an analysis module 301, a misalignment calculation module 303, an impact evaluation module 305, and a recommendation module 307.

In an embodiment, the analysis module 301 may be configured to determine, via the one or more sensors 211 of the wearable device 103 worn by the user 101, the orientation of the head 101a of the user 101 based on an inclination of at least one sensor of the one or more sensors 211. The analysis module 301 may be configured to evaluate a tilt angle (hereinafter, alternatively referred as ‘head tilt angle’) of with the head 101a based on the determined orientation. The head tilt angle may also be called as head flexion and refers to an angle θ at which the user head 101a is rotated with respect to a horizontal axis in relation to the user's body.

The horizontal axis corresponds to a line of sight of the user 101 and refers to an imaginary reference line that extends straight forward and perpendicular to the user's eye. Thus, the line of sight provides a reference that aligns with the user's eye. An operation of the analysis module 301 is described below in greater detail in conjunction with an operation 400B of FIG. 4.

In an embodiment, the misalignment calculation module 303 may be configured to determine a degree of misalignment of the neck 101b based on the head tilt angle. The misalignment calculation module 303 may be configured to determine a force being exerted on the neck 101b based on the head tilt angle and then determine the degree of misalignment based on the force. In an embodiment, the force exerted on the neck 101b corresponds to at least one of a strain, a weight, or a gravitational force exerted on the neck 101b to hold the head 101a stationary in the head tilt angle. The operation of the misalignment calculation module 303 is described below in greater detail in conjunction with an operation 400C of FIG. 4.

In an embodiment, the impact evaluation module 305 may be configured to determine a score associated with a posture of the neck 101b based on a body mass index (BMI) of the user 101, the head tilt angle, the force being exerted on the neck 101b and the degree of misalignment of the neck 101b. The impact evaluation module 305 may be configured to determine whether the posture corresponds to a specific type based on the score. The impact evaluation module 305 may be configured to generate an alert in response to determining that the posture corresponds to the specific type. In an embodiment, the specific type of posture of the neck corresponds to at least one of a good posture, an average posture, and a poor posture.

The impact evaluation module 305 is configured to evaluate a stress level on the neck based on the score. The stress level may refer to the impact of a corresponding score on a section of the neck. The impact evaluation module 305 is configured to determine a duration a corresponding posture may be maintained before causing strain on the neck based on the evaluated stress level. The duration is inversely proportional to the stress level, e.g., the greater the stress level the lesser the duration. Alternatively, the lesser the stress level, the greater the duration. The operation of impact evaluation module 305 is described below in greater detail in conjunction with an operation 400D of FIG. 4.

In an embodiment, the recommendation module 307 may be configured to recommend one or more corrective actions, when the alert associated with the poor posture of the neck 101b is generated. In an embodiment, the one or more corrective actions are recommended in the form of positional feedback, to improve the posture of the neck 101b based on the degree of misalignment of the neck 101b and a stress level on the neck 101b. In an embodiment, the one or more corrective actions may be recommended via at least one of the wearable device or another device communicatively coupled with the wearable device. The operation of recommendation module 307 is described below in greater detail in conjunction with an operation 400E of FIG. 4.

FIG. 4 illustrates a schematic diagram depicting an operational flow of modules, according to an embodiment of the disclosure.

Referring to FIG. 4, in a schematic diagram 400, at an operation 400A, sensor data 401, comprising of both high and low frequency signals, may be collected from the one or more sensors 211 of the wearable device 103 worn by the user 101. In an embodiment, the sensor data 401 may be collected at a predefined sampling rate ‘X’. For example, the sampling rate X predefined as 20 milliseconds indicates that the system 201 is capable of capturing the sensor data at a rate of, approximately 50 samples per second. In an embodiment, the sensor data 401 may be utilized to determine the current body posture of the user 101. For example, the current body posture may correspond to at least one of sitting, standing, and walking. The sensor data 401 may contain noise due to involuntary vibrations and motions during a user activity. Further, accelerometers tend to exhibit positional errors over time and gyroscopes exhibit a steadily growing angular error, which like accelerometers, increases over time.

In an embodiment, the sensor data 401 may be filtered by applying a low pass filter 403 to the sensor data 401. In an embodiment, the low pass filter 403 may be an infinite impulse response (IIR) filter that attenuates high-frequency signals while allowing low-frequency signals to pass through. The IIR filters are widely used to remove noise and unwanted high-frequency components from signals. Thus, the sensor data 401 is filtered to reduce noise, and unwanted high-frequency signals to obtain filtered sensor data 405 for further analysis by the analysis module 301.

In an embodiment, at the operation 400B, the filtered sensor data 405 is provided to the analysis module 301 to determine the orientation 407 of the head 101a. The orientation 407 may be determined based on an inclination of the at least one sensor of the one or more sensors 211 based on a gravity vector and projection of the gravity vector on one or more axes of the at least one sensor. For example, the at least one sensor may be the accelerometer sensor and the one or more axes correspond to the x-axis, y-axis, and z-axis.

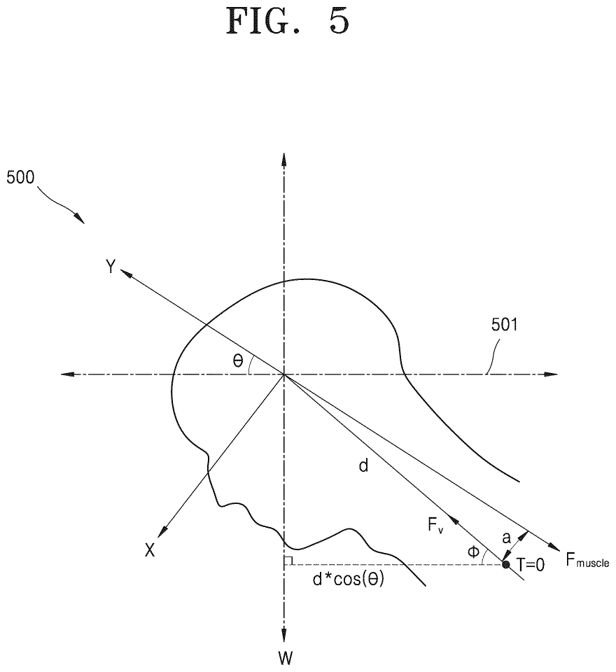

The orientation 407 of the head 101a may be determined based on the inclination of at least one sensor with respect to the gravity vector. The gravity vector may correspond to a horizontal baseline gravity 501, as depicted in FIG. 5.

FIG. 5 illustrates a schematic diagram depicting an orientation of a user's neck and head, according to an embodiment of the disclosure.

Referring to FIG. 5, in a schematic diagram 500, the inclinations of the three axes of at least one sensor are determined with respect to the horizontal axis in degrees. The inclination in the x-axis, y-axis, and z-axis may be determined using Equations 1, 2, and 3 respectively:

$$x - angle ( {⊖}_{x} ) = {sin}^{- 1} ( \frac{x}{\sqrt{{x}^{2} + {y}^{2} + {z}^{2}}} ) \times ( \frac{180}{π} ) degrees \qquad Equation 1$$

$$y - angle ( {⊖}_{y} ) = {sin}^{- 1} ( \frac{y}{\sqrt{{x}^{2} + {y}^{2} + {z}^{2}}} ) \times ( \frac{180}{π} ) degrees \qquad Equation 2$$

$$z - angle ( {⊖}_{z} ) = ( {cos}^{- 1} ( \frac{z}{\sqrt{{x}^{2} + {y}^{2} + {z}^{2}}} ) \times ( \frac{180}{π} ) - 90 ) \times ( - 1 ) degrees \qquad Equation 3$$

The orientation 407 of the head 101a may correspond to one of a forward-flexed orientation or a backward-flexed orientation. In an embodiment, to determine whether the head 101a is flexed forward or backward, a fusion of x-axis and z-axis inclinations may be utilized such that the x-axis of the at least sensor (e.g., accelerometer) points upward while the z-axis coincides with a direction of the user's face.

Thereafter, a tilt angle 409 (denoted as θ) is determined with respect to the line of sight of the user 101, such that the tilt angle 409 corresponds to the inclination of the horizontal axes (e.g., x-axis) of the one or more axes of the at least one sensor (e.g., accelerometer) with respect to the line of sight of the user. The head tilt angle θ is determined such that the magnitude of θ is determined by (\sqrt{x²+y²+z²})>0. Table 1 below depicts values of θ corresponding to inclination of the at least one sensor with respect to the horizontal axis.

TABLE 1

| Inclination | Inclination | Inclination | Head tilt |
|---|---|---|---|
| of x-axis | of y-axis | of z-axis | angle (θ) |
| 9.90960 | −0.66559 | −0.14844 | 86 |
| 8.89925 | 0.36152 | 4.1681 | 64 |
| 6.68222 | −0.22266 | 7.12754 | 43 |

In an embodiment, at operation 400C, the degree of misalignment of the neck 101b is determined by the misalignment calculation module 303 based on the head tilt angle 409. Hereinafter, the term ‘degree of misalignment’ may be used alternatively as ‘neck tilt angle’ (or ‘neck cervical angle’) 413.

In an embodiment, to determine the neck tilt angle 413, the misalignment calculation module 303 determines the force (Fᵥ) 411 being exerted on the neck 101b based on the head tilt angle θ 409. Fᵥ 411 may be exerted by the upper vertebrae of the neck 101b to hold the head 101a stationary. Determination of the Fᵥ 411 is now described in the forthcoming paragraphs.

Fᵥ 411 is determined based on an assumption that said force acts along a line through the center of mass of the user 101 body/head, similar to the force exerted by a gravitational weight (W) of the head 101a and force (F_{muscle}) exerted by neck muscle. Therefore, Fᵥ 411 may also be considered as a gravitational force or strain on the neck 101b. Further, it is considered that W and F_{muscle} are exerted by the head and neck muscles to maintain equilibrium. Therefore, the head 101a is bent at an angle relative to the neck's neutral position.

By a condition for equilibrium at fulcrum T=0, the force exerted at equilibrium (Fₜₒₜₐₗ)=0 at point T=0. Based on a predetermined criterion, said knowledge may be used to determine F_{muscle} when the pivot is chosen to be at the neck (e.g., upper vertebrae). Further, the torque created by W is clockwise, while the torque created by F_{B} (F_{muscle}, the force exerted by the neck muscle) is counterclockwise.

$${F}_{B} \times a = d \times cos ( θ ) \times m \times g \qquad Equation 4$$

where
a=a perpendicular distance between the F_{muscle} and Fᵥ;
d=distance between the gravitational moment (a pivot point around which the neck rotates) of the neck and the center of mass of the head;
m=mass of the neck calculated using a predetermined regression model; and
g=acceleration of gravity.

Therefore:

$${F}_{B} = \frac{d \times cos ( ⊖ ) \times m \times g}{a} \qquad Equation 5$$

In an embodiment, the gravitational moment may be calculated from the perpendicular distance between the vertical axis at the center of gravity (COG) of the head 101a and position C7-T1. C7-T1 is the last part of cervical vertebrae, e.g., the point near the gravitational moment of the neck. In an embodiment, the COG may be estimated as 17% of the distance from a mid-tragus to a vertex of the head 101a. The position C7-T1 may be estimated from the midpoint between a sternal notch marker and a C7 spinous process marker.

Using the y-axis for vertical and x-axis for horizontal, a condition for net external forces along said axes to be zero is defined as

$${F}_{x} = {F}_{B} \times cos ( θ ) \qquad Equation 6$$

$$and$$

$${F}_{y} = W + {F}_{B} \times sin ( θ ) \qquad Equation 7$$

Finally, the resultant force Fᵥ 411 is determined using

$${F}_{v} = \sqrt{F {x}^{2} + F {y}^{2}} \qquad Equation 8$$

and, the direction of the resultant force, e.g., the degree of misalignment of the neck cervical vertebrae (or the neck tilt angle 413) ø is determined as

$$∅ = {tan}^{- 1} ( \frac{{F}_{y}}{{F}_{x}} ) \qquad Equation 9$$

In an embodiment, the gravitational weight W of the head 101a may be 4.17 kg, head tilt angle θ may be 40 degree, and F_{muscle} may be 130.59. Based on the Equations 6 and 7 F_{y} may be determined as 155.24, Fₓ may be determined as 100.03. Finally, based on the Equation 8, Fᵥ is 184.68, and the neck tilt angle ø is 57.20. Table 2 below depicts values of ø for the head tilt angle θ depicted in Table 1.

TABLE 2

| | | | | Force | |
|---|---|---|---|---|---|
| Inclination | Inclination | Inclination | Head tilt | exerted on | Neck tilt |
| of x-axis | of y-axis | of z-axis | angle (θ) | neck (Fᵥ) | angle (ø) |
| 9.90960 | −0.66559 | −0.14844 | 86 | 51.41 | 89.07 |
| 8.89925 | 0.36152 | 4.1681 | 64 | 111.57 | 72.92 |
| 6.68222 | −0.22266 | 7.12754 | 43 | 175.67 | 58.73 |

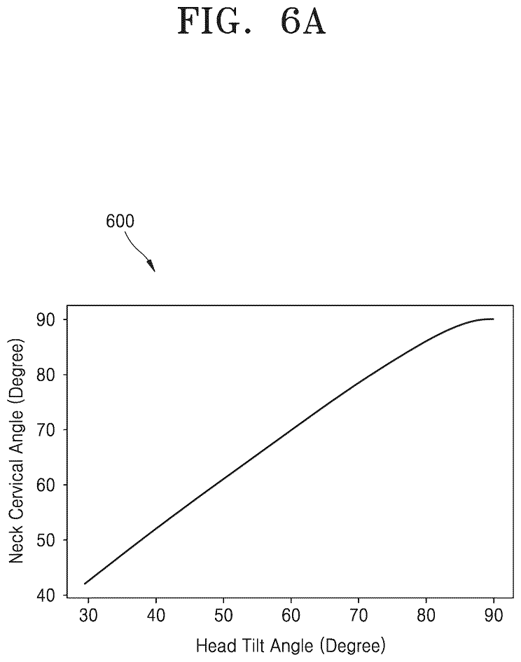

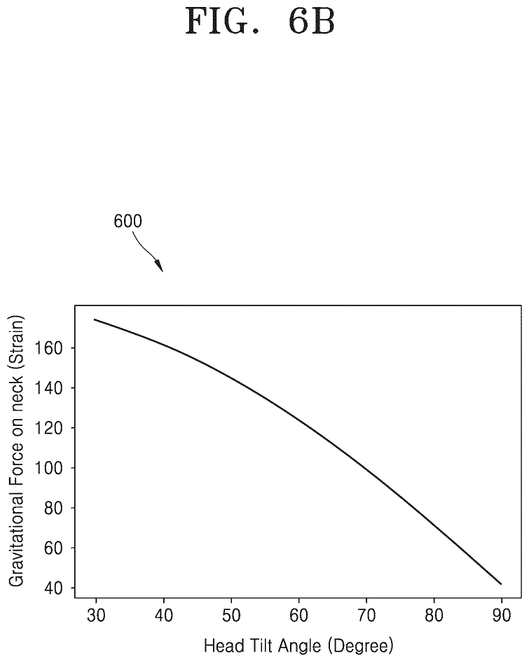

Table 2 can be used to draw a relationship among θ, Fᵥ, and ø, as depicted in FIGS. 6A and 6B.

FIGS. 6A and 6B illustrate graphical diagrams depicting a relationship among a head tilt angle, a gravitational force on the neck, and a neck tilt angle according to an embodiment of the disclosure.

Referring to FIG. 6A, in a graphical diagram 600, when the head 101a is flexed forward, e.g., head 101a is tilted forward, the head tilt angle θ decreases, and the neck tilt angle ø also decreases.

Referring to FIG. 6B, in a graphical diagram 600, when the gravitational force Fᵥ 411 on the neck 101b significantly increases as the head tilt angle θ decreases.

In an embodiment, at operation 400D, the impact evaluation module 305 may determine a score 415 associated with a posture of the neck based on the BMI of the user 101, the head tilt angle (θ) 409, the force (Fᵥ) 411 on the neck 101b, and the degree of misalignment of the neck (the neck tilt angle (ø) 413). In an embodiment, the BMI may be determined using Equation 10 below:

$$BMI = \frac{w}{h \times h} \qquad Equation 10$$

where ‘w’ is the weight of the user 101 in kilograms, and ‘h’ is the height of the user 101 in meters.

In an embodiment, to determine the score 415, values of factors associated with the user, e.g., the BMI, θ, Fᵥ, and ø are normalized such that corresponding values are on the same scale. In an embodiment, the Fᵥ may be normalized based on the head weight of the user 101. A weight is assigned to each factor based on the relative importance of a corresponding factor. The normalized value of each factor is multiplied by a corresponding assigned weight to calculate a weighted score for each factor. The weighted scores for all the factors are summed up to obtain the score 415 associated with the posture of the neck 101b. In an embodiment, the score 415 may be generated in a range between 0 and 1, such that 0 indicates poor posture, while 1 indicates good posture. A scoring is depicted in Table 3 below.

TABLE 3

| Score | 0.961 | 0.611 | 0.10 |
|---|---|---|---|
| Force on the neck, Fᵥ | 48.74 | 89.60 | 198.93 |
| Head tilt angle, θ | 87 | 60 | 35 |
| Neck tilt angle, ø | 89.45 | 73.41 | 54.94 |
| BMI | 23.3 | 23.3 | 23.3 |

In Table 3 above, higher angles with respect to horizontal for both head tilt and neck-cervical angle are given higher scores, indicating that said factors are positively correlated with better posture. On the other hand, higher gravitational force on the neck and longer time duration are associated with poorer posture and given lower scores.

In an embodiment, the score 415 may be utilized to determine the specific type of the posture 417 of the neck. The specific type of the posture 417 may correspond to at least one of a good posture when the score lies in a first predetermined range, an average posture when the score lies in a second predetermined range, and a poor posture when the score lies in a third predetermined range. The impact evaluation module 305 may provide a duration 419 associated with the specific type of posture 417, in response to determining that the posture corresponds to the specific type.

To determine the duration 419, the impact evaluation module 305 may evaluate a stress level on the neck based on the determined score. The stress level may correspond to the impact of a corresponding score on a section of the neck. The section of the neck 101b may refer to a set of cervical vertebrae affected by a corresponding posture of the neck 101b. Based on the stress level, the duration for which the corresponding posture can be maintained before causing strain on the neck may be determined such that the duration is inversely proportional to the stress level. For example, when the stress level is above a threshold stress value, the corresponding posture may be maintained only for a short duration. Similarly, when the stress level is below the threshold stress value, the corresponding posture may be maintained for a longer duration. Evaluation of stress level based on the impact on the section of the neck is depicted in Table 4 below.

TABLE 4

| Head | | | | |
|---|---|---|---|---|
| tilt | Neck tilt | | | Impacted |
| angle | angle | | | section of |
| range | range | Score | | neck |
| (degree) | (degree) | range | Impact on the neck | (vertebrae) |
| 90-76 | 90-84.547 | 1-0.789 | In this position, the load | C1 |
| | | | on the cervical vertebrae | |
| | | | is relatively balanced, | |
| | | | and there is minimal | |
| | | | strain on the | |
| | | | intervertebral discs and | |
| | | | surrounding structures. | |
| | | | C1 vertebra is commonly | |
| | | | affected in this posture | |
| | | | which includes activities | |
| | | | like nodding head etc. | |
| 75-46 | 83.911- | 0.772- | At these angles, the | C2, C3, |
| | 64.441 | 0.275 | pressure on the | C4 |
| | | | intervertebral discs starts | |
| | | | to increase slightly. | |
| | | | Muscles and ligaments | |
| | | | may experience mild | |
| | | | tension and the pressure | |
| | | | on the discs becomes | |
| | | | more noticeable when | |
| | | | the head is bent to a | |
| | | | higher extent of this | |
| | | | range. Under this | |
| | | | condition, C2, C3, and | |
| | | | C4 are the sets of | |
| | | | cervical vertebrae that | |
| | | | are majorly affected. | |
| 45-30 | 63.882- | 0.274- | The strain on muscles | C5, C6 |
| | 57.024 | 0.035 | and ligaments is more | |
| | | | pronounced, leading to | |
| | | | discomfort and potential | |
| | | | musculoskeletal issues. | |
| | | | The second lower sets of | |
| | | | vertebrae e.g. C5 and C6 | |
| | | | may experience higher | |
| | | | damage in this range of | |
| | | | flexion. | |
| Less than | Less than | Less than | Sustaining extreme neck | C6, C7 |
| 30 | 57 | 0.03 | flexion for extended | |
| | | | periods can put immense | |
| | | | stress on the cervical | |
| | | | spine. This can lead to | |
| | | | increased disc pressure, | |
| | | | nerve impingement, and | |
| | | | substantial strain on | |
| | | | muscles and ligaments. | |
| | | | The lower sets of | |
| | | | vertebrae C6 and C7 are | |
| | | | majorly affected due to | |
| | | | this posture. | |

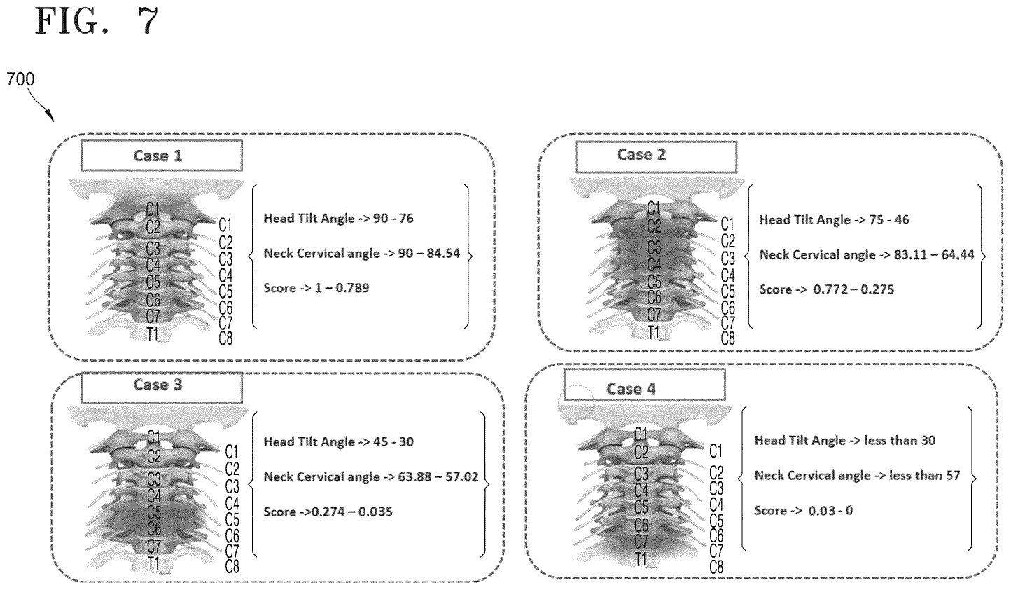

Impact on the sections of the neck as depicted in Table 4 is shown in FIG. 7.

FIG. 7 illustrates a pictorial diagram depicting the impact of a score associated with a posture of a neck on sections of a cervical spine, e.g., the set of vertebrae, according to an embodiment of the disclosure.

Referring to FIG. 7, in a pictorial diagram 700, the impact evaluation module 305 generates an alert 421 in response to determining that the posture corresponds to the specific type. For example, the alert 421 may be generated when the posture of the neck 101b is determined to be either average or poor. In an embodiment, the alert 421 may be generated via at least one of the wearable device 103 or another device (e.g., user device 105) communicatively coupled with the wearable device 103.

In an embodiment, at the operation 400E, the recommendation module 307 provides one or more corrective actions 423 to improve the posture of the neck based on the degree of misalignment of the neck and the stress level on the neck. In an embodiment, the one or more corrective actions 423 may be provided in the form of positional feedback. Table 5 below illustrates one or more corrective actions with respect to the score 415 and the specific type of the posture 417.

TABLE 5

| | Specific | |
|---|---|---|
| Score | type of | |
| range | posture | Corrective actions |
| 1.0-0.514 | Good | No such remarkable requirement of correction in |
| | | the posture. Generally basic movements like |
| | | nodding head, and slightly peeking in a direction. |
| | | The user needs to retract his head by rotating the |
| | | head slightly backward approximately 0-15 |
| | | degrees to maintain this correct posture. |
| | | This posture can be retained for a longer span of |
| | | time as specified for T1 (e.g., no such specific |
| | | range of time) without significantly affecting any |
| | | cervical vertebrae. |
| 0.497-0.274 | Average | Slightly more changes than the good-rated posture |
| | | is required. If no correction is done for a long |
| | | time daily, problems can arise due to repeated |
| | | posture rated as Average. |
| | | The user needs to retract his head by rotating the |
| | | head backward by an approximate angle of 15-30 |
| | | degrees to maintain his posture in neutral posture. |
| | | This posture cannot be maintained for a longer |
| | | duration of time as specified for T2 (e.g., 10-15 |
| | | minutes for a posture) such that T1 > T2. |
| 0.259-0 | Poor | Critically bad postures can cause severe damage |
| | | to the neck if the user doesn't restrain herself |
| | | from being in this posture for a long time. In |
| | | other words, bad-rated postures need to be |
| | | rectified as soon as possible. |
| | | Particular subject has already damaged the |
| | | vertebrae rated subjects need medical |
| | | intervention. |
| | | Immediate actions should be taken to avoid fatal |
| | | neck injury. |
| | | The user needs to retract his head by rotating the |
| | | head backward by an approximate angle of 15-45 |
| | | degrees. |
| | | This posture needs to be corrected immediately as |
| | | specified for time T3 (e.g., less than 1 minutes for |
| | | effective corrections), such that T1 > T2 > T3. |

In an embodiment, the one or more corrective actions 423 may be recommended via at least one of the wearable device 103 or another device (e.g., the user device 105) communicatively coupled with the wearable device 103. In an embodiment, the one or more corrective actions 423 may be recommended as voice output, an image output, or a video output depending on the device on which the one or more corrective actions 423 are recommended and the available interface associated with a corresponding device.

For example, the one or more corrective actions 423 may be given as voice output when provided at the wearable device 103. For example, the one or more corrective actions 423 may be given as image or video output when provided at the user device 105 with a display. Said example may be beneficial when the user 101 is engaged in a call via wearable device 103 and the one or more corrective actions 423 are to be provided. The one or more corrective actions 423 may be provided as an image or video output on another device communicatively coupled with the wearable device and comprises a display.

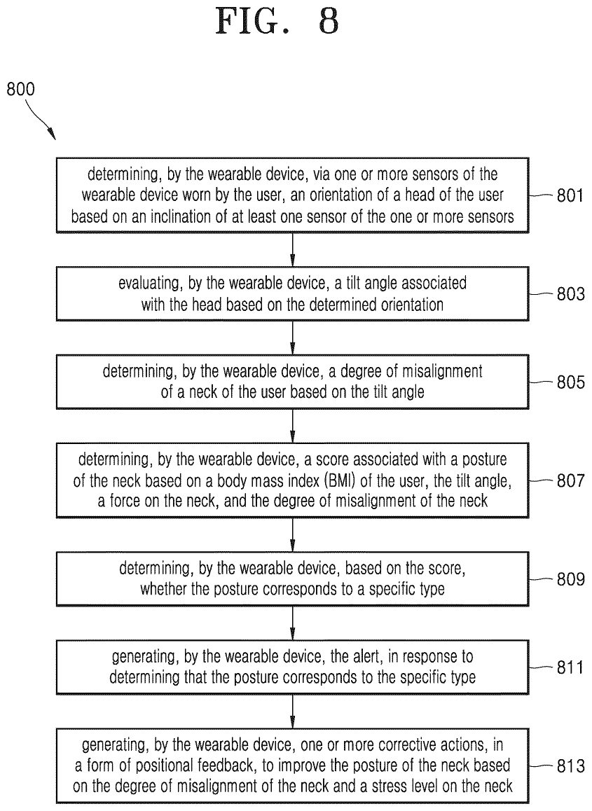

The method 800 for generating the alert based on monitoring of neck alignment of the user is described below in conjunction with FIG. 8.

FIG. 8 illustrates a flow diagram depicting a method for generating an alert based on monitoring of neck alignment of a user, according to an embodiment of the disclosure. A method 800 may include a series of operations 801 through 813 executed by one or more components of the system 201 (e.g., the at least one processor 203).

Referring to FIG. 8, at operation 801, the at least one processor 203 may determine, via the one or more sensors 211 of the wearable device 103 worn by the user 101, an orientation 407 of a head 101a of the user 101 based on an inclination of at least one sensor of the one or more sensors 211. In an embodiment, the processor 203 may determine the inclination of the at least one sensor of the one or more sensors 211 based on a gravity vector and projection of the gravity vector on one or more axes of the at least one sensor. The processor 203 may determine the orientation of the head based on the inclination of the at least one sensor with respect to the gravity vector.

At operation 803, the at least one processor 203 may evaluate the tilt angle 409 associated with the head based on the determined orientation 407. In an embodiment, the processor 203 may determine the tilt angle 409 with respect to a line of sight of the user, wherein the tilt angle 409 corresponds to the inclination of a horizontal axes of the one or more axes of the at least one sensor with respect to the line of sight of the user 101.

At operation 805, the at least one processor 203 may determine a degree of misalignment 413 of the neck 101b of the user 101 based on the tilt angle 409. In an embodiment, determining the degree of misalignment comprises determining a force (Fv) 411 being exerted on the neck based on the tilt angle, and determining the degree of misalignment 413 of the neck based on the determined force (Fv) 411.

At operation 807, the at least one processor 203 may determine the score 415 associated with a posture of the neck 101b based on BMI of the user, the tilt angle 409, the force on the neck 411, and the degree of misalignment 413 of the neck 101b.

At operation 809, the at least one processor 203 may determine, based on the score 415, whether the posture corresponds to the specific type 417. In an embodiment, the specific type of the posture of the neck may correspond to at least one of a good posture when the score lies in a first predetermined range, an average posture when the score lies in a second predetermined range, and a poor posture when the score lies in a third predetermined range. In an embodiment, the processor 203 may provide the duration 419 associated with the specific posture type 417, in response to determining that the posture corresponds to the specific type 417.

In an embodiment, the at least one processor 203 may evaluate the stress level on the neck based on the determined score, the stress level being indicative of an impact of a corresponding score on a section of the neck. The processor 203 may determine the duration, a corresponding posture can be maintained before causing strain on the neck based on the evaluated stress level, wherein the duration is inversely proportional to the stress level.

At operation 811, the at least one processor 203 may generate the alert 421, in response to determining that the posture corresponds to the specific type. In an embodiment, generating the alert may comprise generating the alert to the user via at least one of the wearable device 103 or another device communicatively coupled with the wearable device 103.

At operation 813, the at least one processor 203, in response to generating the alert associated with the average posture or the poor posture of the neck, may recommend one or more corrective actions 423, in a form of positional feedback, to improve the posture of the neck based on the degree of misalignment of the neck and the stress level on the neck. In an embodiment, the at least one processor 203 may recommend the one or more corrective actions 423 via at least one of the wearable device 103 or another device communicatively coupled with the wearable device 103.

At least by virtue of aforesaid, the subject matter at least provides the following advantages.

The method described in an embodiment herein may reduce the usage of external sensors to monitor the neck alignment of the user, thereby improving user experience. The method described in an embodiment herein may generate an alert for correcting neck alignment. The method described in an embodiment herein may auto-balance cervical load on the neck and recommend proper posture when the user is in working mode. The method described in an embodiment herein may recommend proper posture for the particular exercise when the user is in poor or average posture during an exercise session.

It will be appreciated that an embodiment of the disclosure according to the claims and description in the specification can be realized in the form of hardware, software or a combination of hardware and software.

Any such software may be stored in computer readable storage media (e.g. a non-transitory computer-readable storage media). The computer readable storage media store one or more computer programs (software modules), the one or more computer programs include computer-executable instructions that, when executed by one or more processors of an electronic device, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage device and storage medium is an embodiment of machine-readable storage (e.g. a non-transitory machine-readable storage) that are suitable for storing a computer program or computer programs comprising instructions that, when executed, implement an embodiment of the disclosure. Accordingly, an embodiment provides a program comprising code for implementing apparatus or a method as claimed in any one of the claims of this specification and a machine-readable storage storing such a program.

While the disclosure has been shown and describe with reference to an embodiment thereof, it will be understood by those skilled in the art that various changes in form and details may be made therein without departing from the spirit and scope of the disclosure as defined by the appended claims and their equivalents.

In an embodiment, the method may include determining, by the wearable device, the inclination of the at least one sensor of the one or more sensors based on a gravity vector and projection of the gravity vector on one or more axes of the at least one sensor. The method may include determining, by the wearable device, the orientation of the head based on the inclination of the at least one sensor with respect to the gravity vector.

In an embodiment, the method may include evaluating, by the wearable device, the tilt angle with respect to a line of sight of the user. The method, wherein the tilt angle corresponds to the inclination of a horizontal axes of the one or more axes of the at least one sensor with respect to the line of sight of the user.

In an embodiment, the method, wherein determining the degree of misalignment may include determining, by the wearable device, the force being exerted on the neck based on the tilt angle. The method, wherein determining the degree of misalignment may include determining, by the wearable device, the degree of misalignment of the neck based on the determined force.

In an embodiment, the method, wherein the specific type of the posture of the neck correspond to at least one of a good posture when the score lies in a first predetermined range, an average posture when the score lies in a second predetermined range, or a poor posture when the score lies in a third predetermined range.

In an embodiment, the method may include providing, by the wearable device, a duration associated with a specific posture type, in response to determining that the posture corresponds to the specific type.

In an embodiment, the method may include evaluating, by the wearable device, a stress level on the neck based on the determined score, the stress level being indicative of an impact of a corresponding score on a section of the neck. The method may include determining, by the wearable device, the duration, the corresponding posture can be maintained before causing strain on the neck based on the evaluated stress level, wherein the duration is inversely proportional to the stress level.

In an embodiment, the method, wherein generating the alert comprises generating, by the wearable device, the alert to the user via at least one of the wearable device or another device communicatively coupled with the wearable device.

In an embodiment, the method may include in response to generating the alert associated with the posture of the neck, generating, by the wearable device, one or more corrective actions, in a form of positional feedback, to improve the posture of the neck based on the degree of misalignment of the neck and a stress level on the neck.

In an embodiment, the method may include generating, by the wearable device, the one or more corrective actions via at least one of the wearable device or another device communicatively coupled with the wearable device.

In an embodiment, the wearable device, wherein the one or more computer programs further include computer-executable instructions that, when executed by the one or more processors, cause the wearable device to determine the inclination of the at least one sensor of the one or more sensors based on a gravity vector and projection of the gravity vector on one or more axes of the at least one sensor. The one or more processors cause the wearable device to determine the orientation of the head based on the inclination of the at least one sensor with respect to the gravity vector.

In an embodiment, the wearable device, wherein the one or more computer programs further include computer-executable instructions that, when executed by the one or more processors, cause the wearable device to evaluate the tilt angle with respect to a line of sight of the user, wherein the tilt angle corresponds to the inclination of a horizontal axes of the one or more axes of the at least one sensor with respect to the line of sight of the user.

In an embodiment, the wearable device, wherein the one or more computer programs further include computer-executable instructions that, when executed by the one or more processors, cause the wearable device to determine the force being exerted on the neck based on the tilt angle. The one or more processors cause the wearable device to determine the degree of misalignment of the neck based on the determined force.

In an embodiment, the wearable device, wherein the specific type of the posture of the neck corresponds to at least one of a good posture when the score lies in a first predetermined range, an average posture when the score lies in a second predetermined range, or a poor posture when the score lies in a third predetermined range.

In an embodiment, the wearable device, wherein the one or more computer programs further include computer-executable instructions that, when executed by the one or more processors, cause the wearable device to provide a duration associated with a specific posture type, in response to determining that the posture corresponds to the specific type.

In an embodiment, the wearable device, wherein the one or more computer programs further include computer-executable instructions that, when executed by the one or more processors, cause the wearable device to evaluate a stress level on the neck based on the determined score, the stress level being indicative of an impact of a corresponding score on a section of the neck. The one or more processors cause the wearable device to determine the duration, the corresponding posture can be maintained before causing strain on the neck based on the evaluated stress level, wherein the duration is inversely proportional to the stress level.

In an embodiment, the wearable device, wherein generate the alert, the one or more computer programs further include computer-executable instructions that, when executed by the one or more processors, cause the wearable device to generate the alert to the user via at least one of the wearable device or another device communicatively coupled with the wearable device.

In an embodiment, the wearable device, wherein in response to generating the alert associated with the posture of the neck, the one or more computer programs further include computer-executable instructions that, when executed by the one or more processors, cause the wearable device to generate one or more corrective actions, in a form of positional feedback, to improve the posture of the neck based on the degree of misalignment of the neck and a stress level on the neck.

In an embodiment, the wearable device, wherein the one or more computer programs further include computer-executable instructions that, when executed by the one or more processors, cause the wearable device to generate the one or more corrective actions via at least one of the wearable device or another device communicatively coupled with the wearable device.

In an embodiment, the wearable device, wherein the force exerted on the neck is determined based on the force acting along a line through a center of mass of the neck of the user.

In an embodiment, the wearable device, wherein the force exerted on the neck is similar to a force exerted by a gravitational weight of the head and a muscle force exerted by a neck muscle.

In an embodiment, the wearable device, wherein the force exerted on the neck is a gravitational force or strain on the neck.

In an embodiment, the one or more computer-readable storage media, the operations may include determining, by the wearable device, the inclination of the at least one sensor of the one or more sensors based on a gravity vector and projection of the gravity vector on one or more axes of the at least one sensor. The operations may include determining, by the wearable device, the orientation of the head based on the inclination of the at least one sensor with respect to the gravity vector.

In an embodiment, the one or more computer-readable storage media, the operations may include in response to generating the alert associated with the posture of the neck, generating, by the wearable device, one or more corrective actions, in a form of positional feedback, to improve the posture of the neck based on the degree of misalignment of the neck and a stress level on the neck.

What is claimed is:

1. A method performed by a wearable device for generating an alert based on monitoring of neck alignment of a user, the method comprising:
determining, by the wearable device, via one or more sensors of the wearable device worn by the user, an orientation of a head of the user based on an inclination of at least one sensor of the one or more sensors;
evaluating, by the wearable device, a tilt angle associated with the head based on the determined orientation;
determining, by the wearable device, a degree of misalignment of a neck of the user based on the tilt angle;
determining, by the wearable device, a score associated with a posture of the neck based on a body mass index (BMI) of the user, the tilt angle, a force on the neck, and the degree of misalignment of the neck;
determining, by the wearable device, based on the score, whether the posture corresponds to a specific type; and
generating, by the wearable device, the alert, in response to determining that the posture corresponds to the specific type.

2. The method of claim 1, comprising:
determining, by the wearable device, the inclination of the at least one sensor of the one or more sensors based on a gravity vector and projection of the gravity vector on one or more axes of the at least one sensor; and
determining, by the wearable device, the orientation of the head based on the inclination of the at least one sensor with respect to the gravity vector.

3. The method of claim 1, wherein determining the degree of misalignment comprises:
determining, by the wearable device, the force being exerted on the neck based on the tilt angle; and
determining, by the wearable device, the degree of misalignment of the neck based on the determined force.

4. The method of claim 1, wherein the specific type of the posture of the neck correspond to at least one of:
a good posture when the score lies in a first predetermined range;
an average posture when the score lies in a second predetermined range; or
a poor posture when the score lies in a third predetermined range.

5. The method of claim 1, further comprising:
providing, by the wearable device, a duration associated with a specific posture type, in response to determining that the posture corresponds to the specific type.

6. The method of claim 5, further comprising:
evaluating, by the wearable device, a stress level on the neck based on the determined score, the stress level being indicative of an impact of a corresponding score on a section of the neck; and
determining, by the wearable device, the duration, the corresponding posture can be maintained before causing strain on the neck based on the evaluated stress level, wherein the duration is inversely proportional to the stress level.

7. The method of claim 1, wherein generating the alert comprises generating, by the wearable device, the alert to the user via at least one of the wearable device or another device communicatively coupled with the wearable device.

8. The method of claim 1, comprising:
in response to generating the alert associated with the posture of the neck, generating, by the wearable device, one or more corrective actions, in a form of positional feedback, to improve the posture of the neck based on the degree of misalignment of the neck and a stress level on the neck.

9. The method of claim 8, comprising:
generating, by the wearable device, the one or more corrective actions via at least one of the wearable device or another device communicatively coupled with the wearable device.

10. A wearable device for generating an alert based on monitoring of neck alignment of a user, the wearable device comprising:
one or more sensors;
memory storing one or more computer programs; and
one or more processors communicatively coupled to the one or more sensors and the memory,
wherein the one or more computer programs include computer-executable instructions that, when executed by the one or more processors, cause the wearable device to:
determine, via the one or more sensors of the wearable device worn by the user, an orientation of a head of the user based on an inclination of at least one sensor of the one or more sensors,
evaluate a tilt angle associated with the head based on the determined orientation,
determine a degree of misalignment of a neck based on the tilt angle,
determine a score associated with a posture of the neck based on a body mass index (BMI) of the user, the tilt angle, a force on the neck, and the degree of misalignment of the neck,
determine, based on the score, whether the posture corresponds to a specific type, and
generate the alert, in response to determining that the posture corresponds to the specific type.

11. The wearable device of claim 10, wherein the one or more computer programs further include computer-executable instructions that, when executed by the one or more processors, cause the wearable device to:
determine the inclination of the at least one sensor of the one or more sensors based on a gravity vector and projection of the gravity vector on one or more axes of the at least one sensor, and
determine the orientation of the head based on the inclination of the at least one sensor with respect to the gravity vector.

12. The wearable device of claim 10, wherein the one or more computer programs further include computer-executable instructions that, when executed by the one or more processors, cause the wearable device to:
determine the force being exerted on the neck based on the tilt angle, and
determine the degree of misalignment of the neck based on the determined force.

13. The wearable device of claim 10, wherein the specific type of the posture of the neck corresponds to at least one of:
a good posture when the score lies in a first predetermined range;
an average posture when the score lies in a second predetermined range; or
a poor posture when the score lies in a third predetermined range.

14. The wearable device as claimed in claim 10, wherein the one or more computer programs further include computer-executable instructions that, when executed by the one or more processors, cause the wearable device to:
provide a duration associated with a specific posture type, in response to determining that the posture corresponds to the specific type.

15. The wearable device of claim 14, wherein the one or more computer programs further include computer-executable instructions that, when executed by the one or more processors, cause the wearable device to:
evaluate a stress level on the neck based on the determined score, the stress level being indicative of an impact of a corresponding score on a section of the neck, and
determine the duration, the corresponding posture can be maintained before causing strain on the neck based on the evaluated stress level, wherein the duration is inversely proportional to the stress level.

16. The wearable device of claim 10, wherein generate the alert, the one or more computer programs further include computer-executable instructions that, when executed by the one or more processors, cause the wearable device to:
generate the alert to the user via at least one of the wearable device or another device communicatively coupled with the wearable device.

17. The wearable device of claim 10, wherein in response to generating the alert associated with the posture of the neck, the one or more computer programs further include computer-executable instructions that, when executed by the one or more processors, cause the wearable device to:
generate one or more corrective actions, in a form of positional feedback, to improve the posture of the neck based on the degree of misalignment of the neck and a stress level on the neck.

18. The wearable device of claim 17, wherein the one or more computer programs further include computer-executable instructions that, when executed by the one or more processors, cause the wearable device to:
generate the one or more corrective actions via at least one of the wearable device or another device communicatively coupled with the wearable device.

19. One or more non-transitory computer-readable storage media storing one or more computer programs including computer-executable instructions that, when executed by one or more processors of a wearable device, cause the wearable device to perform operations, the operations comprising:
determining, by the wearable device, via one or more sensors of the wearable device worn by a user, an orientation of a head of the user based on an inclination of at least one sensor of the one or more sensors;
evaluating, by the wearable device, a tilt angle associated with the head based on the determined orientation;
determining, by the wearable device, a degree of misalignment of a neck of the user based on the tilt angle;
determining, by the wearable device, a score associated with a posture of the neck based on a body mass index (BMI) of the user, the tilt angle, a force on the neck, and the degree of misalignment of the neck;
determining, by the wearable device, based on the score, whether the posture corresponds to a specific type; and
generating, by the wearable device, an alert, in response to determining that the posture corresponds to the specific type.

20. The one or more non-transitory computer-readable storage media of claim 19, the operations further comprising:
in response to generating the alert associated with the posture of the neck, generating, by the wearable device, one or more corrective actions, in a form of positional feedback, to improve the posture of the neck based on the degree of misalignment of the neck and a stress level on the neck.
